(19) United States
(12) Patent Application Publication
(10) Pub. No.: US 2026/0155218 A1
(43) Pub. Date: Jun. 4, 2026

(54) APPARATUS AND METHOD FOR MODIFYING A VISUALIZATION ASSOCIATED WITH SUBJECT DATA

(71) Applicant: Signet Health Corporation, North Richland Hills, TX (US)

(72) Inventors: Blake Browder, Dallas, TX (US); Joy Figarsky, Little Rock, AR (US)

(73) Assignee: Signet Health Corporation, North Richland Hills, TX (US)

(21) Appl. No.: 18/964,115

(22) Filed: Nov. 29, 2024

(51) Int. Cl. G16H 10/60

(52) U.S. Cl. CPC G16H 10/60

(57) ABSTRACT

An apparatus and method for modifying a visualization associated with subject data. The apparatus includes at least a processor and a memory communicatively connected to the at least a processor. The processor receives subject data associated with at least a subject, analyzes, using an assessment model, the subject data wherein the assessment model is configured to compare the subject data to predefined parameters and determine a variance associated with a comparison between the subject data and the predefined parameters, generates a first visualization of a plurality of visualizations associated with the variance, wherein the first visualization comprises an interaction event handler, displays the first visualization through a graphical user interface, receives an interaction signal through the graphical user interface as a function of the interaction event handler, modifies, using the interaction signal, the first visualization to produce a second visualization, and displays the second visualization through the graphical user interface.

### FIELD OF THE INVENTION

The present invention generally relates to the field of healthcare information systems. In particular, the present invention is directed to an apparatus and a method for modifying a visualization associated with subject data.

### BACKGROUND

Data visualizations are crucial for effectively interpreting and understanding complex datasets. These visualizations serve to provide users with insights by converting raw subject data into graphical representations such as charts, graphs, heatmaps, or other visual forms. However, conventional systems and methods often present visualizations in static or predefined formats, limiting a user's ability to modify, explore, or interact with the subject data in a meaningful way.

### SUMMARY OF THE DISCLOSURE

In an aspect, an apparatus for modifying a visualization associated with subject data includes at least a processor and a memory communicatively connected to the at least a processor. The memory contains instructions configuring the processor to receive subject data associated with at least a subject, analyze, using an assessment model, the subject data wherein the assessment model is configured to compare the subject data to predefined parameters and determine a variance associated with a comparison between the subject data and the predefined parameters, generate, using the at least a processor, a first visualization of a plurality of visualizations associated with the variance, wherein the first visualization comprises an interaction event handler, display, using a downstream device, the first visualization through a graphical user interface, receive, using the at least a processor, an interaction signal through the graphical user interface as a function of the interaction event handler, modify, using the interaction signal, the first visualization to produce a second visualization, and display, using a downstream device, the second visualization through the graphical user interface.

In another aspect, a method for modifying a visualization associated with subject data includes receiving, using at least a processor, subject data associated with at least a subject, analyzing, using an assessment model, the subject data wherein the assessment model is configured to compare the subject data to predefined parameters and determine a variance associated with a comparison between the subject data and the predefined parameters, generating, using the at least a processor, a first visualization of a plurality of visualizations associated with the variance, wherein the first visualization comprises an interaction event handler, displaying, using a downstream device, the first visualization through a graphical user interface, receiving an interaction signal through the graphical user interface as a function of the interaction event handler, modifying, using the interaction signal, the first visualization to produce a second visualization, and displaying, using a downstream device, the second visualization through the graphical user interface.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an apparatus for modifying a visualization associated with subject data;

FIG. 2A is an exemplary illustration of a graphical user interface;

FIG. 2B is an exemplary illustration of a modified graphical user interface;

FIG. 3 is a block diagram of an exemplary machine-learning process;

FIG. 4 is a diagram of an exemplary embodiment of a neural network;

FIG. 5 is a diagram of an exemplary embodiment of a node of a neural network;

FIG. 6 is a block diagram of an exemplary method for modifying a visualization associated with subject data;

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for modifying a visualization associated with subject data. The apparatus includes at least a computing device comprised of a processor and a memory communicatively connected to the processor. The memory instructs the processor to receive subject data associated with at least a subject. The processor analyzes, using an assessment model, the subject data wherein the assessment model is configured to compare the subject data to predefined parameters and determine a variance associated with a comparison between the subject data and the predefined parameters. The processor generate, using the at least a processor, a first visualization of a plurality of visualizations associated with the variance, wherein the first visualization comprises an interaction event handler. Additionally, the processor displays, using a downstream device, the first visualization through a graphical user interface. The processor receives an interaction signal through the graphical user interface as a function of the interaction event handler. The processor modifies, using the interaction signal, the first visualization to produce a second visualization. The processor displays, using a downstream device, the second visualization through the graphical user interface.

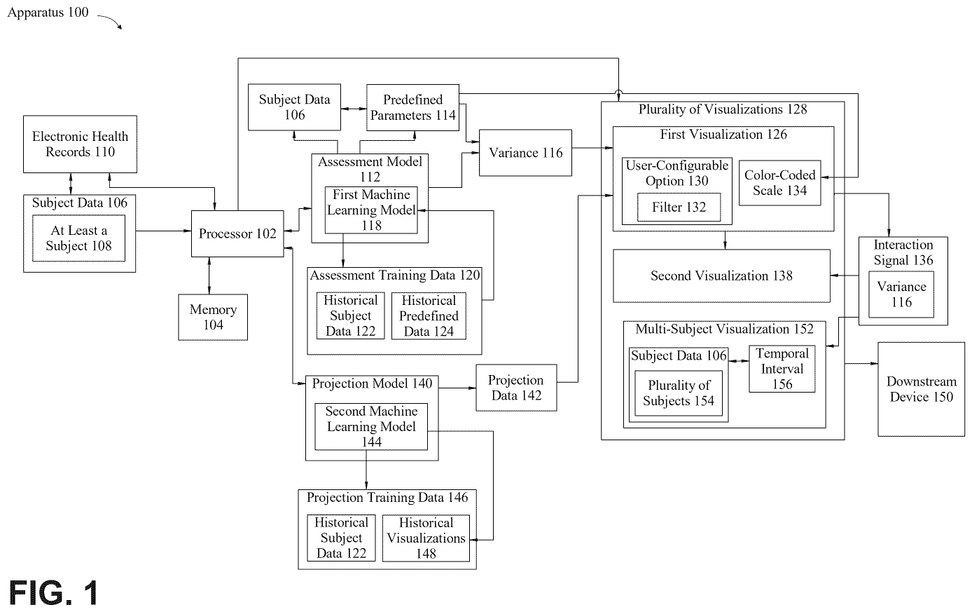

Referring now to FIG. 1, an exemplary embodiment of apparatus 100 for modifying a visualization associated with subject data is illustrated. Apparatus 100 may include a processor 102 communicatively connected to a memory 104. As used in this disclosure, “communicatively connected” means connected by way of a connection, attachment, or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communication connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 104 may include a primary memory and a secondary memory. “Primary memory” also known as “random access memory” (RAM) for the purposes of this disclosure is a short-term storage device in which information is processed. In one or more embodiments, during use of the computing device, instructions and/or information may be transmitted to primary memory wherein information may be processed. In one or more embodiments, information may only be populated within primary memory while a particular software is running. In one or more embodiments, information within primary memory is wiped and/or removed after the computing device has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 102 may access the information from primary memory.

Still referring to FIG. 1, apparatus 100 may include a database. The database may include a remote database. The database may be implemented, without limitation, as a relational database, a key-value retrieval database such as a NOSQL database, or any other format or structure for use as database that a person skilled in the art would recognize as suitable upon review of the entirety of this disclosure. The database may alternatively or additionally be implemented using a distributed data storage protocol and/or data structure, such as a distributed hash table or the like. The database may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 1, apparatus 100 may include and/or be communicatively connected to a server, such as but not limited to, a remote server, a cloud server, a network server and the like. In one or more embodiments, the computing device may be configured to transmit one or more processes to be executed by server. In one or more embodiments, server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, server may be configured to perform one or more processes as described below to allow for increased computational power and/or decreased power usage by the apparatus computing device. In one or more embodiments, computing device may transmit processes to server wherein computing device may conserve power or energy.

Further referring to FIG. 1, apparatus 100 may include any “computing device” as described in this disclosure, including without limitation a microcontroller, microprocessor, digital signal processor (DSP) and/or system on a chip (SoC) as described in this disclosure. Apparatus 100 may include, be included in, and/or communicate with a mobile device such as a mobile telephone or smartphone. Apparatus 100 may include a single computing device operating independently, or may include two or more computing devices operating in concert, in parallel, sequentially or the like; two or more computing devices may be included together in a single computing device or in two or more computing devices. Apparatus 100 may interface or communicate with one or more additional devices as described below in further detail via a network interface device. Network interface device may be utilized for connecting processor 102 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 102 may include but is not limited to, for example, a computing device or cluster of computing devices in a first location and a second computing device or cluster of computing devices in a second location. Apparatus 100 may include one or more computing devices dedicated to data storage, security, distribution of traffic for load balancing, and the like. Apparatus 100 may distribute one or more computing tasks as described below across a plurality of computing devices of computing device, which may operate in parallel, in series, redundantly, or in any other manner used for distribution of tasks or memory between computing devices. Apparatus 100 may be implemented, as a non-limiting example, using a “shared nothing” architecture.

With continued reference to FIG. 1, processor 102 may be designed and/or configured to perform any method, method step, or sequence of method steps in any embodiment described in this disclosure, in any order and with any degree of repetition. For instance, processor 102 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 102 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, processor 102 receives subject data 106 associated with at least a subject 108. As used in this disclosure, “subject data” is data associated with the individual or entity being analyzed. Without limitation, a subject 108 may include a patient. For example, without limitation, the subject 108 may include inpatients in hospitals, long-term care patients, hospice care patients, mental health facility patients, outpatients receiving intensive treatment, post-surgical recovery patients, and the like. In a non-limiting example, subject data 106 may include a patient's demographic details, such as age, gender, and medical history. In another non-limiting example, subject data 106 may include a per diem rate for a specific patient. A per diem rate may include to the daily cost or reimbursement rate associated with that patient's care. Continuing, the per diem rate may vary based on several factors, such as the patient's diagnosis, the level of care required, geographic location, specific treatment protocols, and the like. Continuing, the per diem rate may cover all or some of the services, care, and resources provided to a patient during a 24-hour period. Continuing, the per diem rate may be used in healthcare facilities, insurance reimbursements, research studies to standardize the cost analysis for services provided over a certain period, and the like. Without limitation, the subject data 106 may allow the apparatus to track and analyze the per diem rate of one or more subjects 108 thereby optimizing resource allocation and cost management within healthcare settings. In a non-limiting example, the subject 108 may be admitted to a rehabilitation facility after a knee surgery. Continuing, the facility may charge a per diem rate of $500 per day. Continuing, this per diem rate may include the costs for the subject room, nursing care, physical therapy sessions, medications, and meals. Continuing, if the subject 108 stays at the facility for 10 days, the total charge would be $5,000 based on the per diem rate. In another non-limiting example, the subject 108 may be in hospice care, where a per diem rate might include $230 per day. Continuing, this per diem rat may cover services such as pain management, symptom relief, and emotional support for both the patient and the subject's 108 family.

With continued reference to FIG. 1, the subject data 106 may include real time information, such as real time per diem rates which may reflect the current costs or reimbursements being applied to the patient's ongoing care. Continuing, the real time per diem rates may be particularly useful for dynamic cost-tracking and thereby enabling immediate adjustments based on changes in the subject 108 treatment plan, healthcare facility resources, or market conditions. Alternatively and or additionally, the subject data 106 may include historical information, such as, without limitation, per diem rates. Continuing, the historical per diem rates may allow the apparatus to analyze trends and patterns over time. Continuing, the historical per diem rates may be valuable for retrospective analyses, such as evaluating cost efficiency, forecasting future expenses, or comparing past rates across different treatments and patient demographics.

With continued reference to FIG. 1, the apparatus may be further configured to collect the subject data 106 from electronic health records 110. As used in this disclosure, “electronic health records” are digital records that contain health-related data for an individual. In a non-limiting example, the electronic subject records may be used as a source of subject data 106. In a non-limiting example, an electronic health record may contain a patient's history of allergies, medications, immunization records, and the like, which are accessible to multiple healthcare providers for coordinated care. Without limitation, the electronic health records 110 may include diagnostic imaging results, such as X-rays or MRIs, allowing specialists to review and provide recommendations remotely. In another example, without limitation, the electronic health records 110 may integrate data from multiple sources, including primary care, specialist visits, and emergency room encounters, and the like.

With continued reference to FIG. 1, the apparatus may collect the subject data 106 from the electronic health records 110 by integrating with the healthcare facility's electronic health record system through secure data connections, allowing it to access, retrieve, and analyze relevant patient information. Without limitation, this integration may be achieved using application programming integrations (APIs) or standardized healthcare data formats like HL7 or FHIR, ensuring seamless communication between the electronic health records 110 and the apparatus while maintaining data security and compliance with healthcare regulations like HIPAA.

Still referring to FIG. 1, processor 102 analyzes, using an assessment model 112, the subject data 106 wherein the assessment model 112 is configured to compare the subject data 106 to predefined parameters 114 and determine a variance 116 associated with a comparison between the subject data 106 and the predefined parameters 114. As used in this disclosure, the “assessment model” is a computational or analytical framework that processor 102 uses to evaluate the subject data 106. Without limitation, the assessment model 112 may be configured to compare the subject data 106 against predefined parameters 114. The assessment model 112 may determine a variance 116 based on the comparison.

With continued reference to FIG. 1, as used in this disclosure, “predefined parameters” Without limitation, predefined parameters 114 may include a per diem rate that was previously established by entities responsible for determining the per diem rate. Without limitation, these entities may include healthcare institutions, insurance companies, government agencies, regulatory bodies, and the like. Continuing, the predefined parameters 114 may serve as benchmarks for daily costs or reimbursement rates for patient care, and may be used for comparison against actual data to assess variations in care costs or financial outcomes.

With continued reference to FIG. 1, as used in this disclosure, a “variance” is the measure of deviation between the subject data 106 and the predefined parameters 114. In a non-limiting example, the variance 116 may indicate the amount by which the subjects 108 daily costs are over or under the subject's 108 predefined per diem rate. In another non-limiting example, the variance 116 may quantify the difference between the expected per diem rate and the actual expenses incurred for a subject's 108 care on a given day, providing insights into cost efficiency, potential overages, or savings in patient management. For example, without limiting, a hospital may establish the predefined parameters 114 as a per diem rate of $1,000 for post-surgical patients. Continuing, this predefined parameter may be based on average cost for nursing care, medication, and basic monitoring. Continuing, on a particular day, the subject 108 may incur actual expenses of $1,200 due to unexpected complications requiring additional diagnostic tests and specialized medication. Continuing, the variance 116 for that day would be $200 over the predefined per diem rate, indicating that the patient's care exceeded the expected cost.

With continued reference to FIG. 1, in another non-limiting example, a skilled nursing facility may establish predefined parameters 114, such as, a per diem rate of $300 for rehabilitation patients. Continuing, on a particular day, the subject's 108 actual cost may amount to $250, as the subject 108 required minimal physical therapy and reduced nursing supervision. Continuing, the variance 116 would be −$50 (under the per diem rate), reflecting that the subject's 108 care was less costly than anticipated.

With continued reference to FIG. 1, the assessment model 112 may include a first machine learning model 118 trained on assessment training data 120 comprising historical subject data 122 corresponding to historical predefined data 124. As used in this disclosure, “assessment training data” is a dataset that includes historical subject data 122 and the corresponding historical predefined data 124. Continuing, the assessment training data 120 may be used to train the first machine learning model 118 to identify patterns, relationships, or variances between the subject data 106, such as, actual per diem rates, and the predefined parameters 114, such as the standard per diem rates. Continuing, by analyzing the historical trends, the first machine learning model 118 may learn to recognize deviations, forecast future per diem rates, or evaluate cost-efficiency for new subjects 108 based on their per diem rates compared to the established standards.

With continued reference to FIG. 1, as used in this disclosure, “historical subject data” is past records or information related to a specific subject 108. In a non-limiting example, the historical subject data 122 may include a patient or a set of patients, over a given period. In another non-limiting example, the historical subject data 122 may include the previously recorded per diem costs or reimbursements for a specific patient's care. Continuing, the historical subject data 122 may capture how the daily costs for a patient's care have varied or remained consistent over time.

With continued reference to FIG. 1, as used in this disclosure, “historical predefined data” are the established standard values or benchmarks from past time periods that are used as reference points for comparison. For instance, without limitation, the historical predefined data 124 may include the standard or average per diem rates that were set or expected during previous periods. Continuing, the historical predefined data 124 may be established by entities like healthcare providers, insurers, or regulatory bodies based on policies, historical costs, or average service expenses. In a non-limiting example, the historical subject data 122 may include a patient's per diem rates recorded during a 6-month stay in a skilled nursing facility. For instance, the facility may have recorded daily costs of $350, $375, and $400 over different weeks based on varying levels of care provided to the patient, such as more intensive rehabilitation or specialized nursing support during recovery. Continuing, the historical predefined parameter in this context may be the standard per diem rates established by the skilled nursing facility over the same 6-month period. Continuing, these rates may be set at $360 per day, based on average historical costs and expected services like routine nursing care, rehabilitation, meals, and basic medical supervision. Without limitation, by comparing the historical subject data 122 (the actual per diem costs recorded for the patient) to the historical predefined data 124 (the facility's standard per diem rates), the assessment model 112 may determine whether the patient's care costs were above, below, or in line with the expected standards. Continuing, the comparison may help the facility understand deviations in care costs, identify inefficiencies, or justify higher expenses due to specific patient needs.

Still referring to FIG. 1, processor 102 generates a first visualization 126 of a plurality of visualizations 128 associated with the variance 116, wherein the first visualization 126 includes an interaction event handler. As used in this disclosure, a “visualization” is a representation of data. Without limitation, the visualization may be designed to make information easier to understand and analyze. Without limitation, the variance 116 may be represented through a visualization. In a non-limiting example, the visualization may include charts, graphs, heat maps, and/or other graphical formats. Continuing, the visualization may help a user quickly grasp patterns, trends, anomalies, or relationships within the variance data, aiding in decision-making and strategic planning. For example, without limitation, the visualization may be a line graph or a bar chart showing how a patient's actual daily costs compare to the standard per diem rates over a specific period, highlighting any significant deviations. Additionally and or alternatively, the visualization may include a bar chart representing multiple patients and their respective cost variances, allowing for a comparative analysis of cost efficiency across subjects 108.

With continued reference to FIG. 1, as used in this disclosure, an “event handler,” is a module, data structure, function, and/or routine that performs an action in response to an event. For instance, and without limitation, an event handler may record data corresponding to user selections of previously populated fields such as drop-down lists and/or text auto-complete and/or default entries, data corresponding to user selections of checkboxes, radio buttons, or the like, potentially along with automatically entered data triggered by such selections, user entry of textual data using a keyboard, touchscreen, speech-to-text program, or the like. Event handler may generate prompts for further information, may compare data to validation rules such as requirements that the data in question be entered within certain numerical ranges, and/or may modify data and/or generate warnings to a user in response to such requirements. As used in this disclosure, an “interaction event handler” is a specialized type of event handler focused specifically on user interactions. Without limitation, the interaction event handler may focus on user interaction such as events triggered by direct actions from users, such as clicking a button, swiping, or selecting an item in a list. Continuing, the interaction event handlers may be used to improve user experience by making interfaces responsive and interactive.

With continued reference to FIG. 1, the interaction event handler may be associated with a user-configurable option 130 which may include a filter 132, the interaction signal 136 may include a modification to the filter 132, and modifying the first visualization 126 may include updating the user-configurable option 130, wherein the user-configurable option 130 may include the filter 132. As used in this disclosure, a “user-configurable option” is a setting or feature within the user-interface that allows the user to personalize or adjust the dashboard according to their preferences. In a non-limiting example, the user-configurable option 130 may involve selecting which data to display, choosing specific visual elements, or altering how information is organized. As used in this disclosure, a “filter” is to a tool within the user-interface that allows the user to narrow down or refine the data being displayed. In a non-limiting example, the filter 132 may enable the user to refine the data being displayed by setting specific conditions or criteria. Without limitation, the filter 132 may be applied to focus on particular categories, timeframes, or types of data, helping the user manage large datasets more efficiently. In a non-limiting example, the filter 132 may allow the user to display only log data from a specific date range or filter out certain categories, such as “health” or “financial” data. Continuing, the apparatus may dynamically update the user-interface based on the filter 132 settings, ensuring that only relevant information is shown to the user.

With continued reference to FIG. 1, when a user selects or modifies the filter 132, an event handler detects this action (e.g., a click or selection) and triggers the necessary function to apply the filter 132. Continuing, this allows the user-interface to update in real time, displaying the refined data based on the user's selection. For instance, without limitation, if the user sets the filter 132 to display data from the past week only, the event handler recognizes the user's input, applies the filter criteria, and updates the dashboard to show only relevant data from that time frame. This ensures that the GUI remains responsive and adaptive to the user's configurations, offering a tailored and efficient experience.

With continued reference to FIG. 1, the filter 132 may be used to display only the subjects 108 whose variance 116, such as the per diem rate variance exceeds a certain threshold, or to show subject data 106 from a specific time period or patient demographic. Continuing, the filter 132 may be applied to various types of data to enhance analysis, efficiency, and decision-making by focusing on the most relevant information.

With continued reference to FIG. 1, the interaction event handler may be associated with a color-coded scale 134, the interaction signal 136 may include a modification to the color-coded scale 134, and modifying the first visualization 126 may include updating the color-coded scale 134. As used in this disclosure, a “color-coded scale” is a visual representation that uses different colors to indicate various levels, ranges, or categories of data. In a non-limiting example, the color-coded scale 134 may be based on the predefined parameters 114. Continuing, the color-coded scale 134 may allow users to assign specific colors to correspond with particular values, thresholds, or conditions related to the predefined parameters 114. For example, without limitation, in an application monitoring per diem variances, the color-coded scale 134 may use green to indicate that the variance is within an acceptable range, yellow to signal a moderate deviation, and red to highlight significant deviations from the predefined parameters 114. Continuing, the color-coded scale 134 may provide an intuitive and immediate visual cue for users to understand and assess the data at a glance.

Still referring to FIG. 1, the processor 102 displays, using a downstream device, the first visualization through a graphical user interface. As used in this disclosure, “downstream device” is a device that accesses and interacts with apparatus 100. For instance, and without limitation, downstream device 150 may include a remote device and/or apparatus 100. In a non-limiting embodiment, downstream device 150 may be consistent with a computing device as described in the entirety of this disclosure. In a non-limiting example, the downstream device 150 may include a tablet or smartphone that remotely connects to the apparatus 100 via a network. Continuing the processor 102 may send the first visualization 126 to the tablet, allowing the user to interact with the visualized data on a touchscreen interface. In another non-limiting example, the downstream device 150 may be a desktop computer running a web-based application that accesses the apparatus 100 over a local network. Continuing, the desktop computer may receive the second visualization 138 and provide additional interaction options, such as zooming, filtering, or annotating the displayed data. Continuing the previous non-limiting example, the downstream device 150 may also be a specialized display system, such as an augmented reality headset. Continuing, the headset may display the first visualization 126 in an augmented view, enabling a user to see layered visual data within their physical environment. In an embodiment, without limitation, the downstream device 150 may include a server configured to interact with apparatus 100. The server could further process the visualization data and distribute it to multiple users via their individual devices, such as laptops, allowing collaborative viewing and editing.

Still referring to FIG. 1, processor 102 receives an interaction signal though the graphical user interface as a function of the interaction event handler. As used in this disclosure, an “interaction signal” is an input or command generated by a user's interaction with the system. In a non-limiting example, the interaction signal 136 may prompt the processor to make modifications to a visualization. In another non-limiting example, the interaction signal 136 may be triggered by various actions such as clicking, tapping, swiping, selecting, or adjusting controls (e.g., sliders or buttons) in the user interface.

Still referring to FIG. 1, processor 102 modifies, using the interaction signal 136, the first visualization 126 to produce a second visualization 138. For example, without limitation, in response to the interaction signal 136, such as a user hovering over an icon, within the first visualization 126, processor 102 may automatically display the relevant information in the second visualization 138 without requiring the user to perform the same hovering action repeatedly. Continuing, this interaction signal 136 may allow the apparatus to anticipate user behavior and adjust the graphical user interface (GUI) to present the data in a more accessible or permanent manner, thereby enhancing the user experience by eliminating redundant actions.

With continued reference to FIG. 1, in another non-limiting example, in response to the interaction signal 136, such as a user frequently filtering per diem data by a specific date range, within the first visualization 126, processor 102 may automatically modify the second visualization 138 to display the frequently filtered date range by default. Continuing, the interaction signal 136 may enable the apparatus to recognize the user's repeated preferences and adjust the per diem data in the graphical user interface to present the relevant timeframe consistently, reducing the need for the user to repeatedly apply the same filters.

With continued reference to FIG. 1, the interaction signal 136 may include the variance 116. Without limitation, the interaction signal 136 may be used to trigger a modification or update in the visualization which may be based on the variance 116 between the subject data 106 and the predefined parameters 114. Continuing, the variance 116 may serve as an input or condition that prompts the processor to alter the first visualization 126 and generates the second visualization 138.

With continued reference to FIG. 1, for example, if the variance 116 indicates that the subject data 106, such as the actual per diem costs, exceed the predefined parameters 114, or the per diem rate by a certain threshold, the variance may automatically trigger the interaction signal 136 and cause the processor to modify the first visualization 126 to highlight or expand the details for that particular subject 108 or time period. Continuing, this process may enable the system to respond dynamically based on data changes and automatically adjust the display to draw attention to significant deviations or patterns in the variation datum.

Still referring to FIG. 1, processor 102 displays, using the downstream device 150, the second visualization 138. Without limitation, the processor 102 may create the second visualization 138 by retrieving and processing relevant data, formatting it according to display specifications, and encoding the visualization into a compatible format. Continuing, the downstream device 150 may render the second visualization 138 on its display, allowing users to view and interact with the information.

With continued reference to FIG. 1, the apparatus may further include a projection model 140 configured to generate prediction data 142 of the subject data 106, wherein the prediction data 142 is displayed using the downstream device 150. As used in this disclosure, a “projection model” is a computational or algorithmic framework configured to analyze subject data 106 and generate prediction data 142. Without limitation, the projection model 140 may identify patterns, trends, and/or relationships within the subject data 106. Continuing, the projection model 140 may be designed to forecast future values, behaviors, or outcomes of the subject data 106. In another non-limiting example, the projection model 140 may be configured to analyze the subject data 106 and forecast the likelihood of a subject 108 exceeding the predefined parameters 114, such as the per diem day limit, based on historical trends, patient-specific factors, or real-time data inputs. Continuing, the projection model 140 may consider various attributes, such as a subject's 108 medical history, treatment plans, length of stay, and recovery progress to generate prediction data 142 indicating which patients are at risk of surpassing their per diem allocation.

In a non-limiting example, the projection model 140 may use machine learning techniques to assess past patient records and predict the expected number of per diem days for new or ongoing patients. For instance, without limitation, if a certain treatment type historically leads to extended hospital stays for similar subject 108 profiles, the projection model 140 may flag patients undergoing this treatment as likely to exceed their per diem day limit. Continuing, the prediction data 142 may be displayed on a downstream device 150, such as a medical billing specialist's workstation or a clinician's tablet, to allow early interventions or adjustments to care plans.

With continued reference to FIG. 1, in another non-limiting example, the projection model 140 may be configured to forecast the financial impact of patients going exceeding the predefined parameters 114 or the per diem rates. For instance, without limitation, the projection model 140 may predict which subjects 108 are likely to exceed the per diem limit and estimate the additional costs incurred. Continuing, the prediction data 142 may be visualized on a central dashboard, helping hospital administrators make informed decisions on resource allocation or negotiate rates with payers. Continuing the previous non-limiting example, the projection model 140 may also provide temporal analysis by forecasting how many patients are expected to exceed their per diem days within a specified timeframe, such as the next week or month. Continuing, the projection data 142 may be displayed as a heatmap on a central monitoring system, allowing healthcare managers to proactively allocate resources or adjust patient discharge strategies. In an embodiment, without limitation, the projection model 140 may incorporate seasonal trends or patterns inpatient admissions. For example, without limitation, during flu season, the projection model 140 may predict an increase in the number of patients exceeding their per diem limits due to longer hospital stays. Continuing, the prediction data 142 may be displayed on a downstream device 150 accessible to hospital administrators, enabling strategic planning and budgeting.

With continued reference to FIG. 1, the projection model 140 may include a second machine learning model 144 trained on prediction training data 146 comprising historical subject data 122 corresponding to historical visualizations 148. As used in this disclosure, “prediction training data” is a dataset used to train the projection model 140. In an embodiment, the prediction training data 146 includes historical subject data 122 corresponding to historical visualizations 148. Continuing, the historical subject data 122 corresponding to historical visualizations 148 may serve as the input for the projection model 140 to learn patterns, trends, and relationships that will enable the model to forecast future outcomes or behaviors based on similar subject data 106. As previously mentioned, the historical subject data 122 may include the previously recorded per diem costs or reimbursements for a specific patient's care. As used in this disclosure, “historical visualizations” are modified graphical user interfaces that have been previously used effectively based on interaction signal 136. In a non-limiting example, the historical visualizations 148 may be individual-specific, where the apparatus stores information related to a particular individual or type of individual, along with the graphical user interface configuration that was most effective for viewing the information. Continuing, this ensures that the interface is tailored based on the individual's previous interactions, whether it is a patient, doctor, or another user. Continuing, the historical visualizations 148 may be tailored based on past user interaction data, which tracks how patients engaged with the interface, including what features they frequently accessed or how they interacted with various elements. Without limitation, the projection model 140 may leverage this data to provide insights into which interface designs or layouts were most intuitive for a given patient, allowing future graphical user interfaces to be optimized for ease of use and improved user experience.

With continued reference to FIG. 1, in a non-limiting example, the projection data 142 may be consistent with one or more aspects of an expression of an object described in attorney docket number 1681-029USU1, U.S. patent application Ser. No. 18/964,084, filed on Nov. 29, 2024, titled “SYSTEM AND METHOD FOR DETERMINING AN EXPRESSION OF AN OBJECT,” which is incorporated by reference herein in its entirety.

With continued reference to FIG. 1, displaying a visualization of the plurality of visualizations 128 may include generating a multi-subject visualization 152 configured to display the subject data 106 of a plurality of subjects 154 across a temporal interval 156. As used in this disclosure, a “multi-subject visualization” is a graphical representation configured to simultaneously display subject data 106 from multiple subjects 108. As used in this disclosure, a “temporal interval” is a specific duration of time over which data is observed, collected, or analyzed. Without limitation, the temporal interval 156 may represent a defined period, such as days, weeks, months, or years, within which subject data 106 is tracked or visualized to identify trends, patterns, or changes over time. Continuing, the multi-subject visualization 152 may enable the comparison, analysis, and interpretation of data trends, patterns, or relationships across the plurality of subjects 154 over a defined temporal interval 156. In a non-limiting example, the multi-subject visualization 152 may allow users to simultaneously view and compare per diem data for a plurality of subjects 154, such as patients, across the temporal interval 156, or a specific time period. Without limitation, the graphical representation may be designed to facilitate the comparison, analysis, and interpretation of trends, helping users identify patterns or relationships between per diem spending and factors like patient conditions, treatments, or durations of stay. In a non-limiting example, a hospital administrator may use the multi-subject visualization 152 to track and compare the per diem expenses of several patients over a one-month period. Without limitation, the multi-subject visualization 152 may present each patient's per diem usage in a distinct color or line, showing how their daily spending fluctuates over time. In a non-limiting example, the multi-subject visualization 152 may help the administrator identify subjects who consistently exceed their per diem allowances, allowing them to investigate contributing factors such as treatments or extended stays. Without limitation, a healthcare provider may use the multi-subject visualization 152 to examine the per diem rates of multiple patients discharged within a week. In a non-limiting example, the multi-subject visualization 152 may reveal patterns, such as whether patients undergoing specific surgeries or treatments tend to use more of their per diem budget. Without limitation, this insight may help optimize resource allocation and improve cost management for future patients, such as adjusting treatment strategies or negotiating higher per diem limits with insurers. In a non-limiting example, the multi-subject visualization 152 may allow for historical comparison. Without limitation, a financial manager may overlay current per diem data with historical data from previous months or years, detecting seasonal trends or changes in how per diem funds are allocated. In a non-limiting example, if the historical data shows that flu season correlates with higher per diem usage due to extended hospital stays, the manager could use this insight to plan budgets more effectively.

With continued reference to FIG. 1, the apparatus may further include modifying, using the interaction signal 136, the multi-subject visualization 152. Without limitation, the interface may adapt based on the preferences of individual users. In a non-limiting example, if a doctor or administrator typically prefers to view data for specific patient groups or across certain time frames, the historical visualizations 148 may save these preferences and automatically generate the multi-subject visualization 152 that match their preferred views. Without limitation, this may reduce the need for manual adjustments and streamline the decision-making process, such as when a doctor regularly reviews per diem usage for elderly patients with chronic conditions over a 3-week treatment window.

Without limitation, modifying the multi-subject visualization 152 may help reduce the need for repetitive manual input, streamlining the decision-making process. In a non-limiting example, a doctor who regularly monitors the per diem usage of elderly patients with chronic conditions over a 3-week treatment window may find the system automatically displaying this data configuration each time they log in, without needing to adjust the time frame or select specific patient groups, the doctor can immediately focus on interpreting the data and making informed decisions, thereby increasing workflow efficiency and reducing administrative burdens. Additionally and or alternatively, without limitation, the adaptive approach may assist administrators who need to monitor financial metrics like per diem spending across multiple departments. In a non-limiting example, the apparatus may save their preferences for reviewing spending across different patient groups over specific fiscal periods, enabling the generation of tailored reports that are automatically aligned with their preferred viewing format, ensuring that they quickly access the most pertinent financial data for cost analysis.

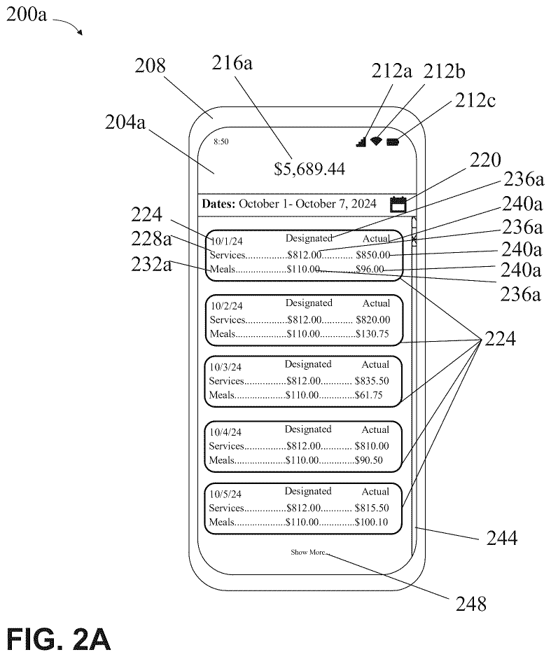

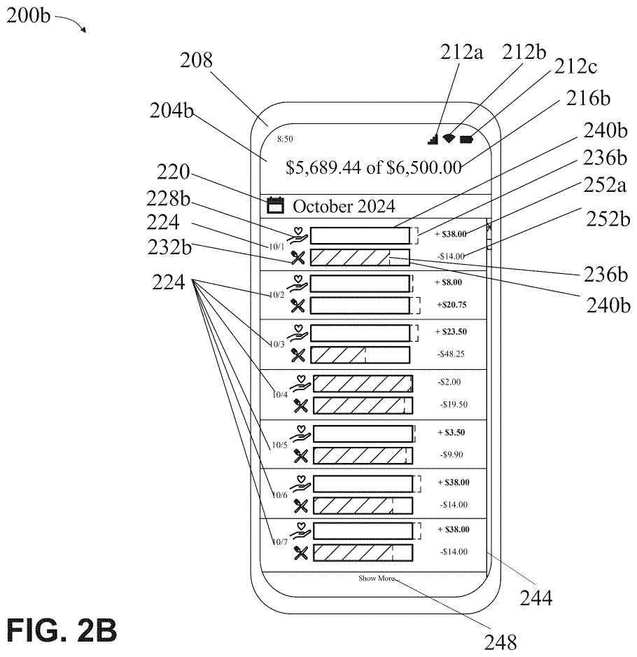

Referring now to FIG. 2A, an exemplary illustration 200a of a graphical user interface. In an embodiment, a graphical user interface 204a is displayed through a downstream device 208. In an embodiment, the graphical user interface 204a includes a signal strength icon 212a, a Wi-Fi icon 212b, a battery icon 212c, and the like. In an embodiment, the signal strength icon 212a displays the strength of the downstream device 208 connection to a mobile network. In an embodiment, the signal strength icon 212a may be depicted as a series of ascending bars, with more bars indicating a stronger signal. Continuing, the signal strength icon 212a may include no bars, representing that the downstream device 208 has little or no connection. In an embodiment, the Wi-Fi icon 212b may include a series of curved lines radiating upwards (like a fan or signal wave). In an embodiment, the Wi-Fi icon 212b may illustrate whether the downstream device 208 is connected to a Wi-Fi network and the strength of the connection. In an embodiment, the battery icon 212c the current battery level of the downstream device 208. In an embodiment, a fully shaded battery may indicate a full charge, while an empty battery icon may indicate that the battery is almost depleted. In an embodiment, battery icon 212c may include additional symbols such as a charging symbol (like a lightning bolt) when the phone is plugged in.

With continued reference to FIG. 2A, in an embodiment, the graphical user interface 204a includes a header 216a. In an embodiment, the header 216a includes subject data, such as, without limitation, the total amount of money spent on the subjects meals and care during a specified time frame in the hospital. In an embodiment, the graphical user interface 204a includes a calendar icon 220. In an embodiment, the calendar icon 220 is an element that visually represents a calendar. Continuing, the calendar icon 220 may be used to allow users to select or view dates within the application. In an embodiment, the calendar icon 220 may triggering date-related functionality when interacted with, such as opening a date picker or scheduling feature.

With continued reference to FIG. 2A, in an embodiment, the graphical user interface 204a includes one or more information windows 224. In an embodiment, the one or more information windows 224 portions of the graphical user interface 204a that provide users with additional details or context about a particular item or function. Continuing, the information window 224 may appear as a small, separate window or overlay when a user interacts with a specific part of the interface, such as clicking, hovering, or tapping on an icon or data point. Without limitation, the one or more information windows 224 may serve as a dynamic, interactive tool within a graphical user interface 204a to provide detailed insights. When a user interacts with a particular date on the calendar icon 220 or schedule view, the information window 224 may display the per diem rate applied to the patient for that specific day, alongside the total amount of per diem benefits already used by the patient up to that point. For example, in a non-limiting scenario, a hospital billing system may feature a calendar where each day corresponds to a patient's stay. When the user hovers over or clicks a specific date, like October 1-Oct. 7, 2024, seven information windows 224 may appear, one for each day. Continuing, the one or more information windows 224 may show the per diem rate charged for that day, as well as how much money the patient has used out of their total per diem allowance. Continuing, the information window 224 may indicate whether the patient is nearing or has exceeded the allocated per diem days, providing real-time feedback in an easy-to-understand format.

With continued reference to FIG. 2A, in an embodiment, the one or more information windows 224 include a service line item 228a, a meals line item 232a, a designated column 236a, and an actual column 240a. In an embodiment, the service line item 228a is a text field within the information window 224 that represents one or more services provided to a subject, such as medical treatment, diagnostic tests, or other billable services. In an embodiment, the meals line item 232a is a text field within the information window 224 that represents meal-related expenses incurred by the subject, such as hospital-provided meals or dietary services. In an embodiment, the designated column 236a is a text field within the information window 224 that displays pre-determined or allocated values, such as planned or budgeted amounts for services or meals, including expected per diem rates or allowances. In an embodiment, the actual column 240a is a text field within the information window 224 that displays the actual values or amounts used, such as the real costs incurred for the services or meals provided, which can be compared to the designated values.

With continued reference to FIG. 2A, in an embodiment, the graphical user interface 204a includes a scroll bar 244. In an embodiment, the scroll bar 244 is an element that allows users to navigate through content that extends beyond the visible area of a window or panel. Continuing, the scroll bar 244 may include of a sliding bar and directional arrows, which users can interact with to move the view horizontally or vertically, enabling access to content that is off-screen. In an embodiment, the graphical user interface 204a may include a “See More . . . ” button 248. In an embodiment, the “See More . . . ” button 248 is an element that allows users to expand or access additional content that is not initially visible. In an embodiment, the when the user clicks or taps the “See More . . . ” button, the interface reveals further details, such as extended information, additional options, or hidden sections, providing a more comprehensive view of the data or functionality within the system.

Referring now to FIG. 2B, an exemplary illustration 200b of a modified graphical user interface. In an embodiment, the modified graphical user interface 204b is displayed through a downstream device 208. In an embodiment, the modified graphical user interface 204b includes a signal strength icon 212a, a Wi-Fi icon 212b, a battery icon 212c, and the like. In an embodiment, the signal strength icon 212a displays the strength of the downstream device 208 connection to a mobile network. In an embodiment, the signal strength icon 212a may be depicted as a series of ascending bars, with more bars indicating a stronger signal. Continuing, the signal strength icon 212a may include no bars, representing that the downstream device 208 has little or no connection. In an embodiment, the Wi-Fi icon 212b may include a series of curved lines radiating upwards (like a fan or signal wave). In an embodiment, the Wi-Fi icon 212b may illustrate whether the downstream device 208 is connected to a Wi-Fi network and the strength of the connection. In an embodiment, the battery icon 212c the current battery level of the downstream device 208. In an embodiment, a fully shaded battery may indicate a full charge, while an empty battery icon may indicate that the battery is almost depleted. In an embodiment, battery icon 212c may include additional symbols such as a charging symbol (like a lightning bolt) when the phone is plugged in.

With continued reference to FIG. 2B, in an embodiment, the modified graphical user interface 204b includes a header 216b. In an embodiment, the header 216b includes subject data, such as, without limitation, the total amount of money spent on the subjects meals and care during a specified time frame in the hospital and the total amount allocated for the specific subject at a given window of time. In an embodiment, the modified graphical user interface 204b includes a calendar icon 220. In an embodiment, the calendar icon 220 is an element that visually represents a calendar. Continuing, the calendar icon 220 may be used to allow users to select or view dates within the application. In an embodiment, the calendar icon 220 may triggering date-related functionality when interacted with, such as opening a date picker or scheduling feature.

With continued reference to FIG. 2B, in an embodiment, the modified graphical user interface 204b includes one or more information windows 224. In an embodiment, the one or more information windows 224 portions of the modified graphical user interface 204b that provide users with additional details or context about a particular item or function. Continuing, the information window 224 may appear as a small, separate window or overlay when a user interacts with a specific part of the interface, such as clicking, hovering, or tapping on an icon or data point. Without limitation, the one or more information windows 224 may serve as a dynamic, interactive tool within the modified graphical user interface 204b to provide detailed insights. When a user interacts with a particular date on the calendar icon 220 or schedule view, the information window 224 may display the per diem rate applied to the patient for that specific day, alongside the total amount of per diem benefits already used by the patient up to that point. For example, in a non-limiting scenario, a hospital billing system may feature a calendar where each day corresponds to a patient's stay. When the user hovers over or clicks a specific date, like October 1-Oct. 7, 2024, seven information windows 224 may appear, one for each day. Continuing, the one or more information windows 224 may show the per diem rate charged for that day, as well as how much money the patient has used out of their total per diem allowance. Continuing, the information window 224 may indicate whether the patient is nearing or has exceeded the allocated per diem days, providing real-time feedback in an easy-to-understand format.

With continued reference to FIG. 2B, in an embodiment, the one or more information windows 224 include a service icon 228b, a meal icon 232b, a designated bar chart 236b, and an actual bar chart 240b. In an embodiment, the service icon 228b is a graphical representation of service within the information window 224 that represents one or more services provided to a subject, such as medical treatment, diagnostic tests, or other billable services. In an embodiment, the meals icon 232b is a graphical representation of food within the information window 224 that represents meal-related expenses incurred by the subject, such as hospital-provided meals or dietary services. In an embodiment, the designated bar chart 236b is a solid line horizontal bar chart within the information window 224 that displays graphically pre-determined or allocated values, such as planned or budgeted amounts for services or meals, including expected per diem rates or allowances. In an embodiment, the actual bar chart 240b is a dashed line horizontal bar chart overlayed on the designated bar chart 236b within the information window 224 that displays the actual values or amounts used, such as the real costs incurred for the services or meals provided, which can be compared to the designated values.

With continued reference to FIG. 2B, in an embodiment, the modified graphical user interface 204b includes a scroll bar 244. In an embodiment, the scroll bar 244 is an element that allows users to navigate through content that extends beyond the visible area of a window or panel. Continuing, the scroll bar 244 may include of a sliding bar and directional arrows, which users can interact with to move the view horizontally or vertically, enabling access to content that is off-screen. In an embodiment, the modified graphical user interface 204b may include a “See More . . . ” button 248. In an embodiment, the “See More . . . ” button 248 is an element that allows users to expand or access additional content that is not initially visible. In an embodiment, the when the user clicks or taps the “See More . . . ” button, the interface reveals further details, such as extended information, additional options, or hidden sections, providing a more comprehensive view of the data or functionality within the system.

With continued reference to FIG. 2B, in an embodiment the modified graphical user interface 204b includes a variance text 252a-b. In an embodiment, the variance text 252a is a text field that displays the amount of money spent over the allocated per diem rate for a given day or service. In an embodiment, the variance text 252a serves as an alert or indicator to users, such as administrators or financial staff, signaling that the subject has exceeded the budgeted per diem allowance. For example, without limitation, if the per diem rate for a patient is $200 and the actual expenses for that day amount to $250, the variance text 252a would display a value of “$50 over,” making it clear that the expenses have surpassed the allowed limit. Continuing, the immediate visibility may help users quickly identify overspending, enabling them to take corrective actions, such as reviewing costs or adjusting care plans.

With continued reference to FIG. 2B, in another embodiment, the variance text 252b shows the amount of money remaining under the per diem rate. In an embodiment, the variance text 252b text field indicates how much of the allocated per diem remains unspent for a particular day or service, providing a quick reference for users to see how much financial flexibility is left. For instance, without limitation, if the per diem rate is $200 and the actual expenses are $150, the variance text 252b would display “$50 left,” allowing the user to understand that there is still room within the budget for additional expenses or services.

With continued reference to FIG. 2B, the modified graphical user interface 204b may present information in a more visually intuitive and user-friendly manner compared to the graphical user interface 204a, by replacing text fields with graphical elements that enhance the user experience through easier data interpretation. In the graphical user interface 204a, service and meal line items are represented by plain text fields, which require users to read and interpret individual entries for each service or expense category. Additionally and or alternatively, the designated and actual values for services and meals are presented in text columns, making it harder for users to immediately grasp comparisons between allocated and actual costs. The modified graphical user interface 204b may improve on this by using icons and visual charts, making the interface more pictorial and intuitive. For example, without limitation, instead of text-based line items, the service and meals are represented by service icons and meal icons, providing an immediate visual cue that simplifies recognition and reduces cognitive load. Continuing, these graphical representations may provide more clarity for users to quickly identify service types or meal-related costs without needing to read through text. Furthermore, the modified graphical user interface 204b may replace the text-based designated and actual columns with bar charts. Continuing, the designated bar chart 240b shows the planned or budgeted amounts as a solid horizontal bar, while the actual bar chart 236b is represented by a dashed line overlaid on the designated bar. Continuing, the visual comparison allows users to instantly see how actual usage compares to the allocated amounts, making the interface far more effective for quick analysis and decision-making. Continuing, the clear visual difference between the two bar charts enables users to easily spot discrepancies or trends at a glance, eliminating the need to compare numeric values manually.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

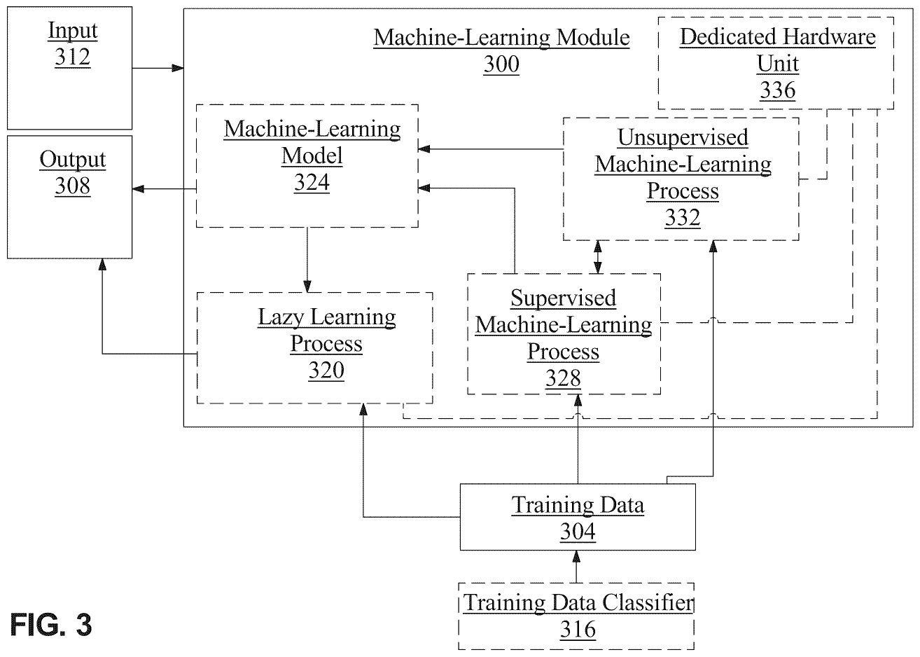

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example inputs may include subject data and predefined parameters and outputs may include variance.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to categories including historical subject data and historical predefined data.

Still referring to FIG. 3, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{Σ}_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 3, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 3, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 3, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 3, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 3, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include subject data and predefined parameters as described above as inputs, variance as output, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 332 may not require a response variable; unsupervised processes 332 may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

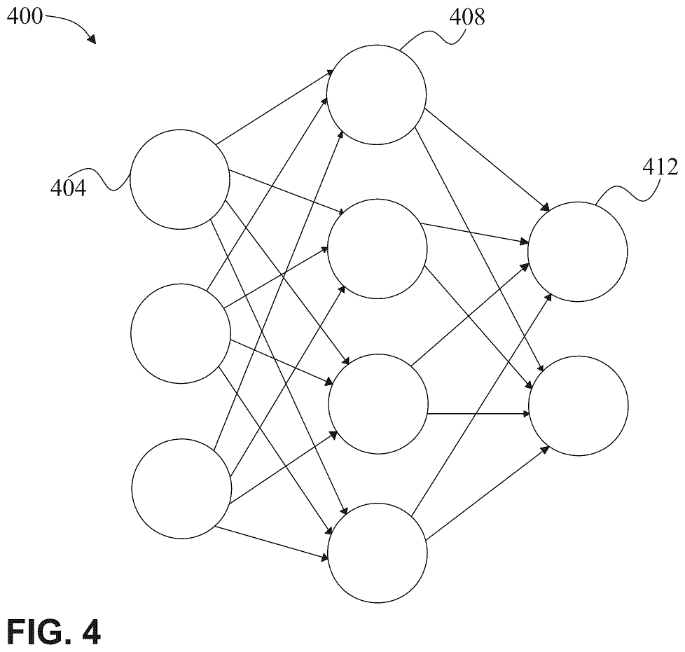

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

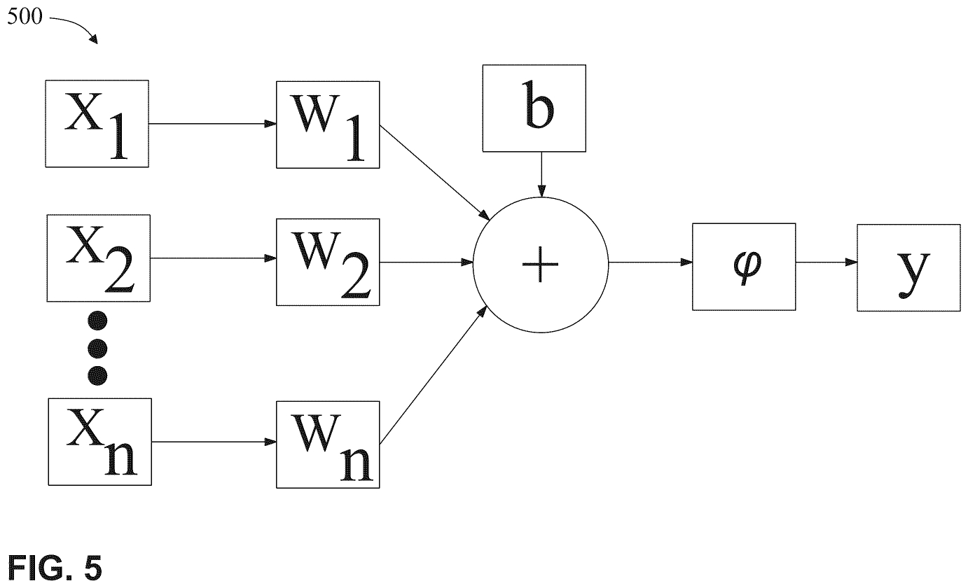

Referring now to FIG. 5, an exemplary embodiment of a node 500 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0,x), a “leaky” and/or “parametric” rectified linear unit function such as f(x)=max(ax,x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{Σ}_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

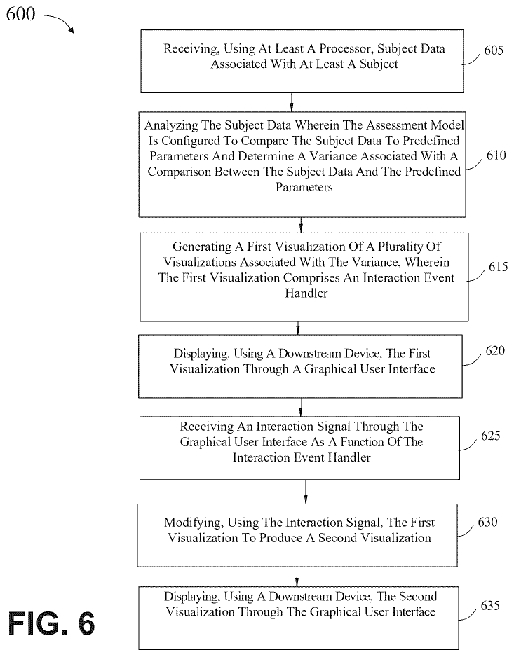

Referring now to FIG. 6, a flow diagram of an exemplary method 600 for modifying a visualization associated with subject data is illustrated. At step 605, method 600 includes receiving, using at least a processor, subject data associated with at least a subject. This may be implemented as described and with reference to FIGS. 1-5.

Still referring to FIG. 6, at step 610, method 600 includes analyzing, using an assessment model, the subject data wherein the assessment model is configured to analyzing, using an assessment model, the subject data wherein the assessment model is configured to compare the subject data to predefined parameters and determine a variance associated with a comparison between the subject data and the predefined parameters. This may be implemented as described and with reference to FIGS. 1-5.

Still referring to FIG. 6, at step 615, method 600 includes generating, using the at least a processor, a first visualization of a plurality of visualizations associated with the variance, wherein the first visualization comprises an interaction event handler. This may be implemented as described and with reference to FIGS. 1-5.

Still referring to FIG. 6, at step 620, method 600 includes displaying, using a downstream device, the first visualization through a graphical user interface. This may be implemented as described and with reference to FIGS. 1-5.

Still referring to FIG. 6, at step 625, method 600 includes receiving an interaction signal through the graphical user interface as a function of the interaction event handler. This may be implemented as described and with reference to FIGS. 1-5.

Still referring to FIG. 6, at step 630, method 600 includes modifying, using the interaction signal, the first visualization to produce a second visualization. This may be implemented as described and with reference to FIGS. 1-5.

Still referring to FIG. 6, at step 635, method 600 includes displaying, using a downstream device, the second visualization through the graphical user interface. This may be implemented as described and with reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

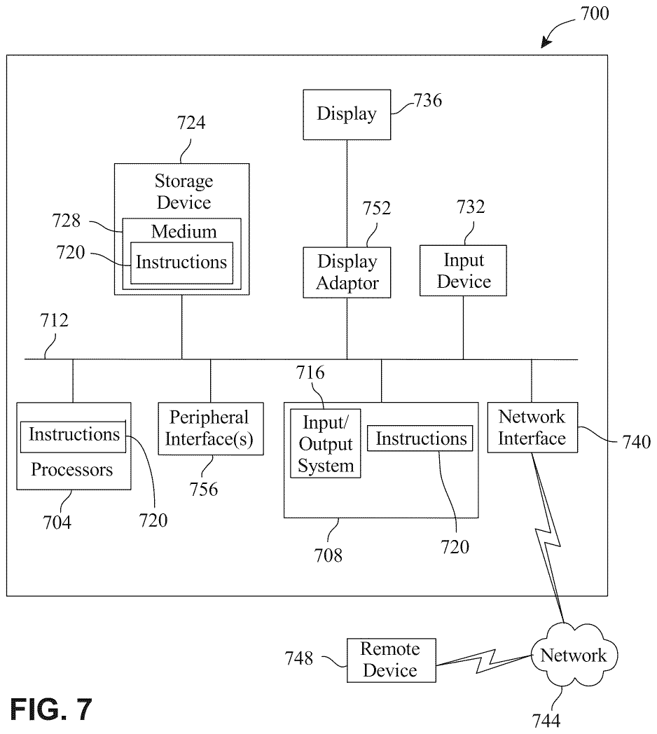

FIG. 7 shows a diagrammatic representation of one embodiment of computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for modifying a visualization associated with subject data, wherein the apparatus comprises:
at least a computing device, wherein the computing device comprises:
a memory; and
at least a processor communicatively connected to the memory, wherein the memory contains instructions configuring the at least a processor to:
receive subject data associated with at least a subject;
analyze, using an assessment model, the subject data wherein:
the assessment model is configured to:
compare the subject data to predefined parameters; and
determine a variance associated with a comparison between the subject data and the predefined parameters; and
the assessment model comprises a first machine-learning model previously trained on assessment training data comprising historical subject data corresponding to historical predefined parameters, wherein training comprises:
upsampling the assessment training data, using at least one of: a set of interpolation rules in order to predict interpolated data associated with the training data; a sample expander method for adding expander data associated with the training data; and a filter for filtering the training data in accordance with a frequency;
downsampling, using a compressor, the training data by removing an nth entry in a sequence of the training data; and
processing the assessment training data by normalizing data entries using feature scaling techniques to ensure the historical predefined parameters are comparable with the subject data received;
generate a first visualization of a plurality of visualizations associated with the variance, wherein the first visualization comprises an interaction event handler associated with at least one of: a module, data structure, function, and routine for performing an action in response to a user interaction;
display, using a downstream device, the first visualization through a graphical user interface;
receive an interaction signal through the graphical user interface as a function of the interaction event handler, wherein the interaction signal is associated with the user interaction and comprises the variance;
modify, using the interaction signal in accordance with the variance, the first visualization to produce a second visualization, wherein modifying the first visualization comprises adjusting the graphical user interface to anticipate subsequent user interactions by changing a default visualization behavior as a function of the variance; and
display, using the downstream device, the second visualization through the graphical user interface.

2. (canceled)

3. The apparatus of claim 1 further comprising a projection model configured to generate projection data of the subject data, wherein the projection data is displayed using the downstream device.

4. The apparatus of claim 3, wherein the projection model comprises a second machine-learning model trained on projection training data comprising historical subject data corresponding to historical visualizations.

5. The apparatus of claim 1, wherein:
the interaction event handler is associated with a user-configurable option comprising a filter;
the interaction signal comprises a modification to the filter; and
modifying the first visualization comprises updating the user-configurable option, wherein the user-configurable option comprises the filter.

6. The apparatus of claim 1, wherein:
the interaction event handler is associated with a color-coded scale;
the interaction signal comprises a modification to the color-coded scale; and
modifying the first visualization comprises updating the color-coded scale.

7. The apparatus of claim 1, wherein displaying the first visualization of the plurality of visualizations comprises generating a multi-subject visualization configured to display the subject data of a plurality of subjects across a temporal interval to enable at least one of comparison, analysis, and interpretation of at least one of a trend, pattern, and relationship.

8. The apparatus of claim 7, wherein the apparatus further comprises modifying, using the interaction signal, the multi-subject visualization.

9. (canceled)

10. The apparatus of claim 1, wherein the apparatus is further configured to collect the subject data from electronic health records.

11. A method for modifying a visualization associated with subject data, wherein the method comprises:
receiving, using at least a processor, subject data associated with at least a subject;
analyzing, using an assessment model, the subject data wherein the assessment model is configured to:
compare the subject data to predefined parameters; and
determine a variance associated with a comparison between the subject data and the predefined parameters;
wherein the assessment model comprises a first machine-learning model previously trained on assessment training data comprising historical subject data corresponding to historical predefined parameters, wherein training comprises:
upsampling the assessment training data, using at least one of: a set of interpolation rules in order to predict interpolated data associated with the training data; a sample expander method for adding expander data associated with the training data; and a filter for filtering the training data in accordance with a frequency;
downsampling, using a compressor, the training data by removing an nth entry in a sequence of the training data; and
processing the assessment training data by normalizing data entries using feature scaling techniques to ensure the historical predefined parameters are comparable with the subject data received;
generating, using the at least a processor, a first visualization of a plurality of visualizations associated with the variance, wherein the first visualization comprises an interaction event handler associated with at least one of: a module, data structure, function, and routine for performing an action in response to a user interaction;
displaying, using a downstream device, the first visualization through a graphical user interface;
receiving, using the at least a processor, an interaction signal through the graphical user interface as a function of the interaction event handler, wherein the interaction signal is associated with the user interaction and comprises the variance;
modifying, using the interaction signal in accordance with the variance, the first visualization to produce a second visualization, wherein modifying the first visualization comprises adjusting the graphical user interface to anticipate subsequent user interactions by changing a default visualization behavior as a function of the variance; and
displaying, using the downstream device, the second visualization through the graphical user interface.

12. (canceled)

13. The method of claim 11 further comprising a projection model configured to generate projection data of the subject data, wherein the projection data is displayed using the downstream device.

14. The method of claim 13, wherein the projection model comprises a second machine-learning model trained on projection training data comprising historical subject data corresponding to historical visualizations.

15. The method of claim 11, wherein: the interaction event handler is associated with a user-configurable option comprising a filter;
the interaction signal comprises a modification to the filter; and
modifying the first visualization comprises updating the user-configurable option, wherein the user-configurable option comprises the filter.

16. The method of claim 11, wherein:
the interaction event handler is associated with a color-coded scale;
the interaction signal comprises a modification to the color-coded scale; and
modifying the first visualization comprises updating the color-coded scale.

17. The method of claim 11, wherein displaying the first visualization of the plurality of visualizations comprises generating a multi-subject visualization configured to display the subject data of a plurality of subjects across a temporal interval to enable at least one of comparison, analysis, and interpretation of at least one of a trend, pattern, and relationship.

18. The method of claim 17, further comprising modifying, using the interaction signal, the multi-subject visualization.

19. (canceled)

20. The method of claim 11, further comprising collecting, by the at least a processor, the subject data from electronic health records.
